(19) United States
(12) Patent Application Publication
(10) Pub. No.: US 2023/0355921 A1
(43) Pub. Date: Nov. 9, 2023

(54) Sleep Aid Device

(71) Applicant: Pillow Cube, Inc., Lindon, UT (US)

(72) Inventor: Jay Davis, Lindon, UT (US)

(21) Appl. No.: 18/314,114

(22) Filed: May 8, 2023

Related U.S. Application Data
(60) Provisional application No. 63/338,944, filed on May 6, 2022.
(60) Provisional application No. 63/402,394, filed on Aug. 30, 2022.

(51) Int. Cl. A61M 21/02

(52) U.S. Cl. CPC A61M 21/02; A61M 2205/3306; A61M 2205/3368; A61M 2205/3375; A61M 2021/0027

(57) ABSTRACT

A sleep aid system includes a computing device, a plurality of monitoring sensors coupled to the computing device, and one or more stimulation devices coupled to the computing device. The plurality of monitoring sensors may be configured to monitor a user before and during sleep of the user. The one or more stimulation devices are configured to produce one or more stimuli. The computing device processes inputs from the plurality of monitoring sensors and activates the one or more stimulation devices to produce the one or more stimuli based on the processed inputs.

### CROSS-REFERENCE TO RELATED APPLICATIONS

This application claims the benefit of U.S. Provisional Application No. 63/338,944, filed on May 6, 2022, which is incorporated by reference herein in its entirety. This application also claims the benefit of U.S. Provisional Application No. 63/402,394, filed on Aug. 30, 2022, which is incorporated by reference herein in its entirety.

### FIELD

This disclosure relates generally to a sleep aid system. More particularly, embodiments of the invention relate to a sleep aid system with monitoring sensors and stimulation devices. Embodiments of the invention relate to a sleep aid system that may be configured to provide stimulation to a user based on data collected and analyzed.

### BACKGROUND

Proper sleep is important for individuals. Proper environment before, during, and after sleep may not only maintain but may improve one's physical well-being. In addition, proper sleeping environment may help one to avoid over-stressing their body. Data from sleep may prove useful to individuals and help them to have a better overall sleep environment. When analyzing sleep, it is possible that different people respond differently to the same stimuli. While overall patterns may help, individualized sleep stimuli may allow a user to dial in a sleep environment especially geared toward their body. Embodiments described herein allow for a sleep aid system to be used to reach better and more restful sleep. Embodiments herein allow for consistent and or tailored sleep environment and also allow a user to wake up refreshed and rested without soreness or stiffness.

The subject matter of the present application has been developed in response to the present state of the art, and in particular, in response to the problems and disadvantages associated with conventional systems that have not yet been fully solved by currently available techniques. Accordingly, the subject matter of the present application has been developed to provide embodiments of a system, apparatus, and method that overcome at least some of the shortcomings of prior art techniques.

### SUMMARY

Disclosed herein is a sleep aid system. The sleep aid system includes a computing device, a plurality of monitoring sensors coupled to the computing device, and one or more stimulation devices coupled to the computing device. The plurality of monitoring sensors may be configured to monitor a user before and during sleep of the user. The one or more stimulation devices are configured to produce one or more stimuli. The computing device processes inputs from the plurality of monitoring sensors and activates the one or more stimulation devices to produce the one or more stimuli based on the processed inputs. The preceding subject matter of this paragraph characterizes example 1 of the present disclosure.

The plurality of monitoring sensors comprises a wearable device on the user, wherein the wearable device is connected to the computing device. The preceding subject matter of this paragraph characterizes example 2 of the present disclosure, wherein example 2 also includes the subject matter according to example 1, above.

The plurality of monitoring sensors comprises a temperature sensor, an audio reception device, and a camera. The preceding subject matter of this paragraph characterizes example 3 of the present disclosure, wherein example 3 also includes the subject matter according to any one of examples 1-2, above.

The one or more stimulation devices includes a speaker configured to emit sounds. The preceding subject matter of this paragraph characterizes example 4 of the present disclosure, wherein example 4 also includes the subject matter according to any one of examples 1-3, above.

The one or more stimulation devices includes a scent dispenser configured to emit scents. The preceding subject matter of this paragraph characterizes example 5 of the present disclosure, wherein example 5 also includes the subject matter according to any one of examples 1-4, above.

The one or more stimulation devices includes one or more lights configured to produce different sequence or intensity of lights for the user. The preceding subject matter of this paragraph characterizes example 6 of the present disclosure, wherein example 6 also includes the subject matter according to any one of examples 1-5, above.

The computing device is connected to a thermostat and the computing device is further configured to adjust the temperature based on the inputs from the plurality of monitoring sensors. The preceding subject matter of this paragraph characterizes example 7 of the present disclosure, wherein example 7 also includes the subject matter according to any one of examples 1-6, above.

The computing device is further configured to adjust the lights based on the inputs from the plurality of monitoring sensors. The preceding subject matter of this paragraph characterizes example 8 of the present disclosure, wherein example 8 also includes the subject matter according to any one of examples 1-7, above.

The computing device is further configured to adjust audio output from the speaker based on the inputs from the plurality of monitoring sensors. The preceding subject matter of this paragraph characterizes example 9 of the present disclosure, wherein example 9 also includes the subject matter according to any one of examples 1-8, above.

The plurality of monitoring sensors are configured to record and monitor the sleep pattern of the user and match the sleep pattern to monitored conditions in a room where the user sleeps. The preceding subject matter of this paragraph characterizes example 10 of the present disclosure, wherein example 10 also includes the subject matter according to any one of examples 1-9, above.

The monitored conditions include a temperature and an amount of light in the room. The preceding subject matter of this paragraph characterizes example 11 of the present disclosure, wherein example 11 also includes the subject matter according to any one of examples 1-10, above.

The computing device is configured to run a sequence of stimuli matched to a sleep pattern of the user. The preceding subject matter of this paragraph characterizes example 12 of the present disclosure, wherein example 12 also includes the subject matter according to any one of examples 1-11, above.

The computing device sends data collected from the user to a cloud connected computing device, wherein the cloud computing device includes an artificial intelligence (AI) algorithm that monitors the inputs and stimuli and determines a personalized stimuli sequence for the user. The preceding subject matter of this paragraph characterizes example 13 of the present disclosure, wherein example 13 also includes the subject matter according to any one of examples 1-12, above.

The computing device is configured to run a wake up sequence of stimuli to wake a user from sleep. The preceding subject matter of this paragraph characterizes example 14 of the present disclosure, wherein example 14 also includes the subject matter according to any one of examples 1-13, above.

Disclosed herein is a sleep aid system. The sleep aid system includes a computing device, a plurality of monitoring sensors coupled to the computing device and configured to monitor a user before and during sleep of the user, wherein the plurality of monitoring sensors includes wearable device on the user, a temperature sensor, an audio reception device, or a camera, and one or more stimulation devices coupled to the computing device, wherein the one or more stimulation devices are configured to produce one or more stimuli. The computing device processes inputs from the plurality of monitoring sensors and activates the one or more stimulation devices to produce the one or more stimuli based on the processed inputs. The preceding subject matter of this paragraph characterizes example 15 of the present disclosure.

The sleep aid system further includes a cloud computing device connected to the computing device. The preceding subject matter of this paragraph characterizes example 16 of the present disclosure, wherein example 16 also includes the subject matter according to example 15, above.

The computing device sends data collected from the user to the cloud connected computing device, wherein the cloud computing device includes an artificial intelligence (AI) algorithm that monitors the inputs and stimuli and determines a personalized stimuli sequence for the user. The preceding subject matter of this paragraph characterizes example 17 of the present disclosure, wherein example 17 also includes the subject matter according to any one of examples 15-16, above.

The computing device is connected to a thermostat and the computing device is further configured to adjust the temperature based on the inputs from the plurality of monitoring sensors. The preceding subject matter of this paragraph characterizes example 18 of the present disclosure, wherein example 18 also includes the subject matter according to any one of examples 15-17, above.

The one or more stimulation devices includes one or more lights configured to produce different sequence or intensity of lights for the user, and wherein the computing device is further configured to adjust the lights based on the inputs from the plurality of monitoring sensors. The preceding subject matter of this paragraph characterizes example 19 of the present disclosure, wherein example 19 also includes the subject matter according to any one of examples 15-18, above.

The one or more stimulation devices includes a speaker configured to emit sounds, and wherein the computing device is further configured to adjust audio output from the speaker based on the inputs from the plurality of monitoring sensors. The preceding subject matter of this paragraph characterizes example 20 of the present disclosure, wherein example 20 also includes the subject matter according to any one of examples 15-19, above.

Other aspects and advantages of embodiments of the present invention will become apparent from the following detailed description, taken in conjunction with the accompanying drawings, illustrated by way of example of the principles of the invention.

### BRIEF DESCRIPTION OF DRAWINGS

In order that the advantages of the subject matter may be more readily understood, a more particular description of the subject matter briefly described above will be rendered by reference to specific embodiments that are illustrated in the appended drawings. Understanding that these drawings depict only typical embodiments of the subject matter and are not therefore to be considered limiting of its scope, the subject matter will be described and explained with additional specificity and detail through the use of the drawings.

FIG. 1 depicts a schematic diagram of a computing system for a sleep aid device according to one or more embodiments of the present disclosure.

FIG. 2 depicts a schematic diagram of a sleep aid device according to one or more embodiments of the present disclosure.

FIG. 3 depicts a perspective view of a sleep aid system, according to one or more embodiments of the invention.

FIG. 4 depicts a schematic diagram of a method, according to one or more embodiments of the invention.

Throughout the description, similar reference numbers may be used to identify similar elements. The following list is an example of the reference numbers used in the accompanying drawings:

| Reference # | Designation |
|---|---|
| 200 | Sleep Aid Device |
| 12 | Computer System |
| 14 | Processing Unit |
| 16 | Bus |
| 18 | I/O Interface(s) |
| 20 | Network Adaptor(s) |
| 22 | Memory |
| 24 | RAM |
| 26 | Cache Memory |
| 28 | Storage Media |
| 110 | Control System |
| 122 | Sensors |
| 210 | Monitoring Sensors |
| 240 | Modal Engine |
| 270 | Stimulation Devices |
| 272 | Speaker |
| 274 | Light or LEDs |
| 276 | Diffuser or Dispenser |
| 278 | Thermometer |

Throughout this application, similar designations or vocabulary may be used to identify similar elements, although the breadth of this disclosure should be understood to incorporate any alternatives and variations referenced within the specification (including the claims) and the accompanying drawings.

### DETAILED DESCRIPTION

It will be readily understood that the components of the embodiments as generally described herein and illustrated in the appended figures could be arranged and designed in a wide variety of different configurations. Thus, the following more detailed description of various embodiments, as represented in the figures, is not intended to limit the scope of the present disclosure but is merely representative of various embodiments. While the various aspects of the embodiments are presented in drawings, the drawings are not necessarily drawn to scale unless specifically indicated.

The present invention may be embodied in other specific forms without departing from its spirit or essential characteristics. The described embodiments are to be considered in all respects only as illustrative and not restrictive. The scope of the invention is, therefore, indicated by the appended claims rather than by this detailed description. All changes which come within the meaning and range of equivalency of the claims are to be embraced within their scope.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussions of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize, in light of the description herein, that the invention can be practiced without one or more of the specific features or advantages of a particular embodiment. In other instances, additional features and advantages may be recognized in certain embodiments that may not be present in all embodiments of the invention.

Reference throughout this specification to “one embodiment,” “an embodiment,” or similar language means that a particular feature, structure, or characteristic described in connection with the indicated embodiment is included in at least one embodiment of the present invention. Thus, the phrases “in one embodiment,” “in an embodiment,” and similar language throughout this specification may, but do not necessarily, all refer to the same embodiment.

While many embodiments are described herein, at least some of the described embodiments describe a sleep aid device. In at least some embodiments, the sleep aid device includes various features and functions that aid a person during a sleep cycle. Some embodiments include monitoring devices, processing devices, and stimulation devices.

While many embodiments are described herein, at least some of the described embodiments describe a sleep aid system. In an illustrative embodiment, the sleep aid system includes a computing device, a plurality of monitoring sensors coupled to the computing device, and one or more stimulation devices coupled to the computing device. The plurality of monitoring sensors may be configured to monitor a user before and during sleep of the user. The one or more stimulation devices are configured to produce one or more stimuli. The computing device processes inputs from the plurality of monitoring sensors and activates the one or more stimulation devices to produce the one or more stimuli based on the processed inputs.

In some embodiments, the plurality of monitoring sensors comprises a wearable device on the user, wherein the wearable device is connected to the computing device.

In some embodiments, the plurality of monitoring sensors comprises a temperature sensor, an audio reception device, and a camera.

In some embodiments, the one or more stimulation devices includes a speaker configured to emit sounds.

In some embodiments, the one or more stimulation devices includes a scent dispenser configured to emit scents.

In some embodiments, the one or more stimulation devices includes one or more lights configured to produce different sequence or intensity of lights for the user.

In some embodiments, the computing device is connected to a thermostat and the computing device is further configured to adjust the temperature based on the inputs from the plurality of monitoring sensors.

In some embodiments, the computing device is further configured to adjust the lights based on the inputs from the plurality of monitoring sensors.

In some embodiments, the computing device is further configured to adjust audio output from the speaker based on the inputs from the plurality of monitoring sensors.

In some embodiments, the plurality of monitoring sensors are configured to record and monitor the sleep pattern of the user and match the sleep pattern to monitored conditions in a room where the user sleeps.

In some embodiments, the monitored conditions include a temperature and an amount of light in the room.

In some embodiments, the computing device is configured to run a sequence of stimuli matched to a sleep pattern of the user.

In some embodiments, the computing device sends data collected from the user to a cloud connected computing device, wherein the cloud computing device includes an artificial intelligence (AI) algorithm that monitors the inputs and stimuli and determines a personalized stimuli sequence for the user.

In some embodiments, the computing device is configured to run a wake up sequence of stimuli to wake a user from sleep.

Disclosed herein is a sleep aid system. The sleep aid system includes a computing device, a plurality of monitoring sensors coupled to the computing device and configured to monitor a user before and during sleep of the user, wherein the plurality of monitoring sensors includes wearable device on the user, a temperature sensor, an audio reception device, or a camera, and one or more stimulation devices coupled to the computing device, wherein the one or more stimulation devices are configured to produce one or more stimuli. The computing device processes inputs from the plurality of monitoring sensors and activates the one or more stimulation devices to produce the one or more stimuli based on the processed inputs.

In some embodiments, the sleep aid system further includes a cloud computing device connected to the computing device.

In some embodiments, the computing device sends data collected from the user to the cloud connected computing device, wherein the cloud computing device includes an artificial intelligence (AI) algorithm that monitors the inputs and stimuli and determines a personalized stimuli sequence for the user.

In some embodiments, the computing device is connected to a thermostat and the computing device is further configured to adjust the temperature based on the inputs from the plurality of monitoring sensors.

In some embodiments, the one or more stimulation devices includes one or more lights configured to produce different sequence or intensity of lights for the user, and wherein the computing device is further configured to adjust the lights based on the inputs from the plurality of monitoring sensors.

In some embodiments, the one or more stimulation devices includes a speaker configured to emit sounds, and wherein the computing device is further configured to adjust audio output from the speaker based on the inputs from the plurality of monitoring sensors.

Computing System

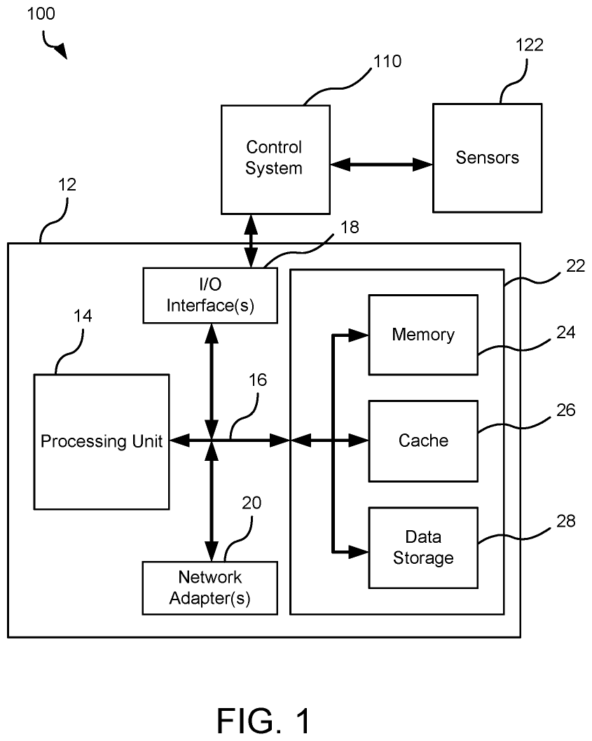

Referring to FIG. 1, a schematic diagram of a computing system 100 is shown. Although the computing system 100 is shown and described with certain components and functionality, other embodiments of the computing system 100 may include fewer or more components to implement less or more functionality.

In some embodiments, aspects of the computing system 100 are implemented via a networked system or a computer system 12 or its component parts. The illustrated computer system 12 may include, but is not limited to, one or more processing arrangements, for example including processors or processing units 14, a communication bus 16, one or more input/output (I/O) adapters 18, one or more network adapters 20, and a system memory 22.

In one embodiment, the system memory 22 includes computer system readable media in the form of volatile memory, such as random-access memory (RAM) 24 and/or cache memory 26. The system memory 22 may further include other removable/non-removable, volatile/non-volatile computer system storage media 28 In such instances, each can be connected to the bus 16 by one or more data media interfaces. The memory 22 may include at least one program product having a set (e.g., at least one) of program modules that are configured to carry out the functions of proposed embodiments. For instance, the memory 22 may include a computer program product having program executable by the processing unit 14 to perform processes described herein. Programs and/or utilities having a set (at least one) of program modules may be stored in the memory 22. Program modules generally carry out the functions and/or methodologies described herein.

The computer system 12 may also communicate with one or more external devices such as a keyboard, a display, sensors 122, cameras, apps, or other external devices, including but not limited to a control system 110. Also, the computer system 12 can communicate with one or more networks such as a local area network (LAN), a general wide area network (WAN), and/or a public network (e.g., the Internet) via network adapter 20.

In the context of the present application, where embodiments of the present invention constitute a method, it should be understood that such a method is a process for execution by a computer, i.e. is a computer-implementable method. The steps of the method therefore reflect various parts of a computer program, e.g., parts of one or more algorithms. Embodiments of the present invention may be a system, a method, and/or a computer program product. The computer program product may include a computer readable storage medium (or media) having computer readable program instructions thereon for causing a processor to carry out aspects of the present invention.

Referring to FIG. 1 again, the control system 110 interacts with and receives signals from sensors 80. The control system 110 is further configured to control the system 100 and its function. In some embodiments, more than one control system 110 may control the various components of the system 100.

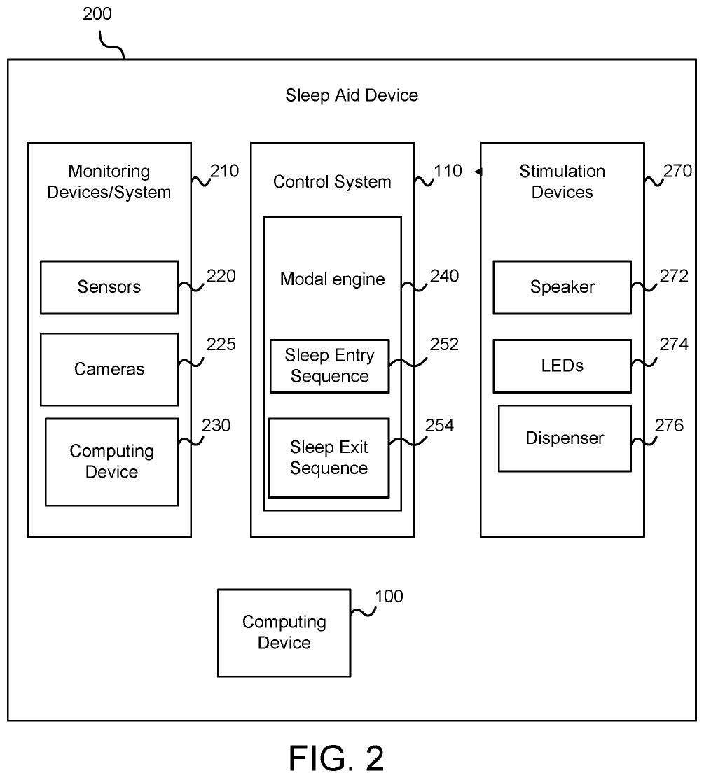

Referring to FIG. 2, a schematic diagram of a sleep aid device is shown. The sleep aid device 200 includes a computing device 100, monitoring sensors 210, a modal engine 240, and stimulation devices 270. Although the sleep aid device 200 is shown and described with certain components and functionality, other embodiments of the sleep aid device 200 may include fewer or more components to implement less or more functionality.

In some embodiments, the monitoring system 210 includes sensors 220, cameras, 225, and a computing device 230 (similar to the computing device of FIG. 1 or a separate device or system that is external and connected by the cloud). The control system 110 includes the modal engine 240 which may include a sleep entry sequence 252 and a sleep exit sequence 254. The stimulation devices 270 may include a speaker 272, LEDs 274, and a dispenser 276.

In some embodiments, the plurality of monitoring sensors may include variations or permutations of many different monitoring sensors or devices. In some embodiments, the plurality of monitoring sensors may include cameras. The cameras can pick up visual cues from the user or the environment.

In some embodiments, the plurality of monitoring sensors may include microphones. The microphone(s) may pick up auditory cues such as breathing patterns or snoring etc.

In some embodiments, the plurality of monitoring sensors may include connected wearable devices (Bluetooth and Wi-Fi connected). This would encompass many different types of wearable devices including watches etc. These devices may be able to monitor vital signs and further sleep cues to be able to monitor when a user has entered deep sleep etc.

In some embodiments, the plurality of monitoring sensors may include LIDAR. In some embodiments, the LIDAR can be a peripheral sensor-addon. In some embodiments, it can be separately attached to a headboard or other area in the room. The LIDAR may also pick up various sleeping cues from the user.

In some embodiments, the plurality of monitoring sensors may include motion detectors. The motion detectors may be able to detect the movement of a user to know whether a restless sleep is occurring among other cues.

In some embodiments, the plurality of monitoring sensors may include temperature sensors or thermometers. Knowing the temperature of the room in which the individual sleeps can be helpful to know whether the person tends to sleep better at a certain temperature or not.

In some embodiments, the plurality of monitoring sensors may include sensors that monitor air flow and composition of the air.

Some embodiments monitor the sleeping of a user. Monitoring may include, but is not limited to, the monitoring of a person's heart rate, breathing, sleep position, and physical movement. The monitoring system may also track and store sleep information including, but not limited to, duration of sleep, sleep times, duration of REM sleep, etc.

In addition, the sleep aid device may monitor the room including noise levels or other sounds in the environment. The sleep aid device 200 may monitor the temperature of the room throughout the sleeping period. The sleep aid device 200 may also monitor wearable connected devices to gather data on the user.

All data may be anonymized but tied to a specific user. Such data can allow for the sleep aid device to learn how best to aid the sleep of the user by determining among other things, what the best temperature of the room might be, what lights work best to enter deep sleep, what sounds work best on the user. As all users may be different, such data may be unique and allow the sleep aid device 200 to determine the best course of action for each unique user.

In some embodiments, the sleep aid system includes a computing device, a plurality of monitoring sensors coupled to the computing device and configured to monitor a user before and during sleep of the user, wherein the plurality of monitoring sensors includes wearable device on the user, a temperature sensor, an audio reception device, or a camera, and one or more stimulation devices coupled to the computing device, wherein the one or more stimulation devices are configured to produce one or more stimuli. The computing device processes inputs from the plurality of monitoring sensors and activates the one or more stimulation devices to produce the one or more stimuli based on the processed inputs.

In some embodiments, the sleep aid system further includes a cloud computing device connected to the computing device. In some embodiments, the computing device sends data collected from the user to the cloud connected computing device, wherein the cloud computing device includes an artificial intelligence (AI) algorithm that monitors the inputs and stimuli and determines a personalized stimuli sequence for the user. In some embodiments, the computing device is connected to a thermostat and the computing device is further configured to adjust the temperature based on the inputs from the plurality of monitoring sensors.

In some embodiments, the one or more stimulation devices includes one or more lights configured to produce different sequence or intensity of lights for the user, and wherein the computing device is further configured to adjust the lights based on the inputs from the plurality of monitoring sensors. In some embodiments, the one or more stimulation devices includes a speaker configured to emit sounds, and wherein the computing device is further configured to adjust audio output from the speaker based on the inputs from the plurality of monitoring sensors.

Referring again to FIG. 2, the sleep aid device may include a modal engine 240. Some embodiments include a connectivity option that allows the sleep information and mode information to be synced with a fitness monitor or other fitness profile to incorporate sleep information into an overall fitness profile.

Embodiments of the sleep aid device include various features that are configured to be activated in response to the monitored state of the user. Various stimuli may be activated to aid a person in going to sleep, staying asleep, and/or waking up.

The modal engine 240 may be able to run personalized sequences particular to a user or sequences known to help other users. These sequences can be run before, during, or after sleep or after a particular cue is picked up by the plurality of monitoring sensors. That is, if the monitoring sensors notice that a user is not in a restful sleep, the modal engine 240 may be able to run particularized stimuli. This may include changing the temperature or turning on a fan or initiating another stimuli (which are discussed herein in detail). There may be a nighttime sequence, a morning sequence (or wake up sequence), a REM cycle sequence, etc.

In some embodiments, the sleep aid device utilizes AI algorithms that can learn and adapt sequences or stimuli as needed. The AI can learn from the data gathered to tailor the best experience for a user. In some embodiments, the sleep aid device may monitor a user to determine whether certain fragrances help a user to sleep. The sleep aid device may determine what temperature leads to the deepest sleep of the user. Different variables can be tested and over time the AI, in conjunction with an algorithm, may determine different sequences that can be used to help the user fall asleep, stay asleep, or wake up in the best manner.

As an example, if a user consistently wakes up at a certain time of the night, the sleep aid device and AI can determine whether any changes in the environment may lead to a user staying in deep sleep longer. As another example, the sleep aid device and the AI can determine whether there are factors that lead a user to fall asleep more quickly by simulating a sunset through lighting or stimulating the user through fragrances or sounds.

AI unique personal and environmental issues-data can be uploaded to the cloud to interface with the AI. In some embodiments, the AI only exists in the cloud and not within each sleep aid device allowing for the computing apparatus on the sleep aid device to not be overwhelmed with data and a constantly progressing AI.

Referring back to FIG. 2, there are various stimulation devices that may be coupled to the computing device.

In some embodiments, the one or more stimulation devices may include lights. This may include LEDs or other types of lights and may including the ability to do various operations of the lights in the room or environment, not limited to dimming, strobing, or changing colors.

In some embodiments, the one or more stimulation devices may include dispensers. The dispensers may be able to dispense humidified air in some embodiments. The dispensers may be able to dispense dehumidified air in some embodiments. The dispensers may be able to dispense scents or aromas that are geared to the user for sleep.

In some embodiments, the one or more stimulation devices may include a speaker. The speaker may be able to play songs, music, sleep sounds, or other things that are able to relax a user or wake them if needed.

In some embodiments, the one or more stimulation devices may include an internet connected device or other appliance or function of the house such as lighting or air conditioning such that the sleep aid device can initiate the internet connected device to start or otherwise perform a needed task.

Some embodiments of the invention include various modes that are triggered by the monitoring of a user, that trigger the stimuli to be activated. The sleep aid device can determine what stimuli most aid a person to enter a sleep state, return to a sleep state, or exit a sleep state. The sleep aid device can monitor the movement of the user, the breathing pattern, snoring, etc. to determine what type of sleep state a user is in and trigger a mode to activate to aid the user in their sleep cycle.

In some embodiments, the sleep aid device is connected to sleep monitoring wearables. The sleep aid device can also be connected to an App on a phone to allow a user to interface with the sleep aid device on their phone. Such interface allows for other inputs to be entered. Such data may include a user rating their overall sleep satisfaction on a certain night. Such satisfaction data could be compared to observable data on what temperature it was, what fragrances were used, what lighting was used.

In some embodiments, the sleep aid device utilizes preprogrammed sleep and wake routines based on machine learning of optimal sleeping and waking environments. In some embodiments, custom sleep and wake routines can be entered or developed.

In some embodiments, the sleep aid device utilizes aromas to stimulate the user. Some embodiments include an aromatherapy diffuser to diffuse or dispense various scents when falling asleep or when waking from sleep.

In some embodiments, the sleep aid device utilizes different sounds and noises to best aid sleep. White noise or water or other sounds can be used at various points to help the user fall asleep, stay asleep, or wake up from sleep.

In some embodiments, the sleep aid device utilizes a microphone to allow for hands-free voice control of the sleep aid device. The voice control allows for hands free control of the devices features and settings.

In some embodiments, the sleep aid device utilizes light control to allow for optimal sleep for the user. Automatic adjustments to the lighting can prepare the user for sleep, help aid them in falling asleep, staying asleep, or waking from sleep.

In some embodiments, the sleep aid device can be used to control smart devices within the room and your house. In some embodiments, the sleep aid device can adjust the temperature of a thermostat or the lights or other smart devices that are connected to the internet.

Some embodiments utilize machine learning to learn from the data that is collected to best optimize the sleep aid device and optimize sleep patterns for the user. The machine learning can learn what sound, light, temperature, and scent adjustments lead to the best sleeping result.

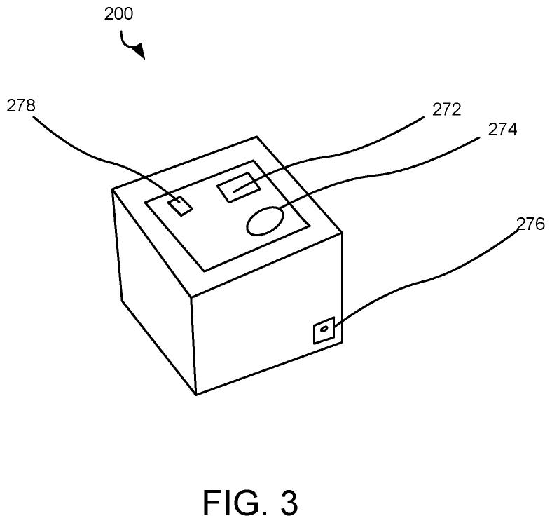

Referring to FIG. 3, a sleep aid device 200 is depicted. The sleep aid device 200 of FIG. 3 may include some or all the features discussed herein. The illustrated embodiment includes a speaker 272, a light 274, a dispenser or diffuser 276, and a thermometer 278 to monitor the temperature of the room. The sleep aid device may interact with other wearable devices and smart devices near the user. The sleep aid device may also interact with AI or machine learning through the cloud to optimize sleep patterns for the user.

Allowing the majority of processing to occur in the cloud allows for the sleep aid device to be smaller and more geared towards the user without wasting processer time at the device.

In some embodiments, a sleep aid system includes a computing device, a plurality of monitoring sensors coupled to the computing device, and one or more stimulation devices coupled to the computing device. The plurality of monitoring sensors may be configured to monitor a user before and during sleep of the user. The one or more stimulation devices are configured to produce one or more stimuli. The computing device processes inputs from the plurality of monitoring sensors and activates the one or more stimulation devices to produce the one or more stimuli based on the processed inputs.

In some embodiments, the plurality of monitoring sensors comprises a wearable device on the user, wherein the wearable device is connected to the computing device. In some embodiments, the plurality of monitoring sensors comprises a temperature sensor, an audio reception device, and a camera. In some embodiments, the one or more stimulation devices includes a speaker configured to emit sounds. In some embodiments, the one or more stimulation devices includes a scent dispenser configured to emit scents.

In some embodiments, the one or more stimulation devices includes one or more lights configured to produce different sequence or intensity of lights for the user. In some embodiments, the computing device is connected to a thermostat and the computing device is further configured to adjust the temperature based on the inputs from the plurality of monitoring sensors. In some embodiments, the computing device is further configured to adjust the lights based on the inputs from the plurality of monitoring sensors. In some embodiments, the computing device is further configured to adjust audio output from the speaker based on the inputs from the plurality of monitoring sensors.

In some embodiments, the plurality of monitoring sensors are configured to record and monitor the sleep pattern of the user and match the sleep pattern to monitored conditions in a room where the user sleeps. In some embodiments, the monitored conditions include a temperature and an amount of light in the room. In some embodiments, the computing device is configured to run a sequence of stimuli matched to a sleep pattern of the user. In some embodiments, the computing device sends data collected from the user to a cloud connected computing device, wherein the cloud computing device includes an artificial intelligence (AI) algorithm that monitors the inputs and stimuli and determines a personalized stimuli sequence for the user. In some embodiments, the computing device is configured to run a wake up sequence of stimuli to wake a user from sleep.

In some embodiments, the sleep aid system includes a computing device, a plurality of monitoring sensors coupled to the computing device and configured to monitor a user before and during sleep of the user, wherein the plurality of monitoring sensors includes wearable device on the user, a temperature sensor, an audio reception device, or a camera, and one or more stimulation devices coupled to the computing device, wherein the one or more stimulation devices are configured to produce one or more stimuli. The computing device processes inputs from the plurality of monitoring sensors and activates the one or more stimulation devices to produce the one or more stimuli based on the processed inputs.

In some embodiments, the sleep aid system further includes a cloud computing device connected to the computing device. In some embodiments, the computing device sends data collected from the user to the cloud connected computing device, wherein the cloud computing device includes an artificial intelligence (AI) algorithm that monitors the inputs and stimuli and determines a personalized stimuli sequence for the user.

In some embodiments, the computing device is connected to a thermostat and the computing device is further configured to adjust the temperature based on the inputs from the plurality of monitoring sensors. In some embodiments, the one or more stimulation devices includes one or more lights configured to produce different sequence or intensity of lights for the user, and wherein the computing device is further configured to adjust the lights based on the inputs from the plurality of monitoring sensors. In some embodiments, the one or more stimulation devices includes a speaker configured to emit sounds, and wherein the computing device is further configured to adjust audio output from the speaker based on the inputs from the plurality of monitoring sensors.

Methods of using the sleep aid system are contemplated herein.

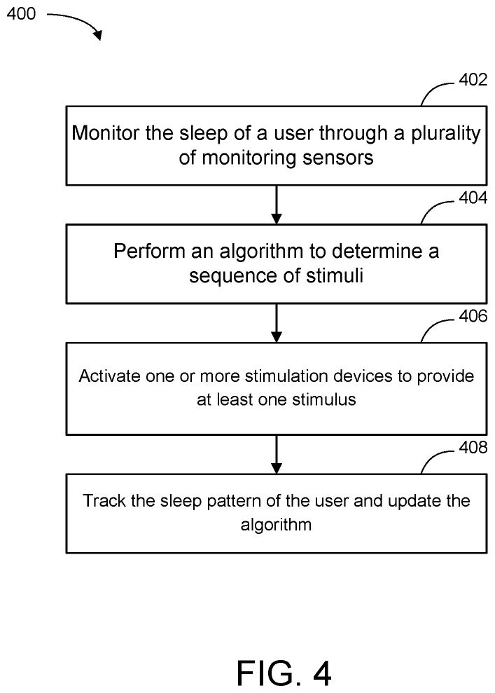

Referring to FIG. 4, a method 400 for initiating a sleep aid device is disclosed. At block 402, the method 400 includes monitoring the sleep of a user through a plurality of monitoring sensors. At block 404, the method 400 includes initiating and performing an algorithm to determine a sequence of stimuli. At block 406, the method 400 includes activating one or more stimulation devices to provide at least one stimulus. At block 408, the method 400 includes tracking the sleep pattern of the user and updating the algorithm. The method 400 then ends.

The method may include various changes or alterations to the above such that the monitoring that has been discussed herein may be utilized. In addition, the various stimuli discussed herein may be used including using various sequences that might be used. In some embodiments, the method may include utilizing AI algorithms to adjust or help home in the sequence used by the sleep aid device. These variations are not repeated herein for the sake of brevity.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

Reference throughout this specification to features, advantages, or similar language does not imply that all of the features and advantages that may be realized with the subject matter of the present disclosure should be or are in any single embodiment. Rather, language referring to the features and advantages is understood to mean that a specific feature, advantage, or characteristic described in connection with an embodiment is included in at least one embodiment of the present disclosure. Thus, discussion of the features and advantages, and similar language, throughout this specification may, but do not necessarily, refer to the same embodiment.

In the above description, specific details of various embodiments are provided. However, some embodiments may be practiced with less than all of these specific details. In other instances, certain methods, procedures, components, structures, and/or functions are described in no more detail than to enable the various embodiments of the invention, for the sake of brevity and clarity.

Although the operations of the method(s) herein are shown and described in a particular order, the order of the operations of each method may be altered so that certain operations may be performed in an inverse order or so that certain operations may be performed, at least in part, concurrently with other operations. In another embodiment, instructions or sub-operations of distinct operations may be implemented in an intermittent and/or alternating manner.

In the above description, certain terms may be used such as “up,” “down,” “upper,” “lower,” “horizontal,” “vertical,” “left,” “right,” and the like. These terms are used, where applicable, to provide some clarity of description when dealing with relative relationships. But, these terms are not intended to imply absolute relationships, positions, and/or orientations. For example, with respect to an object, an “upper” surface can become a “lower” surface simply by turning the object over. Nevertheless, it is still the same object. Further, the terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

Although specific embodiments of the invention have been described and illustrated, the invention is not to be limited to the specific forms or arrangements of parts so described and illustrated. The scope of the invention is to be defined by the claims appended hereto and their equivalents.

As used herein, the phrase “at least one of”, when used with a list of items, means different combinations of one or more of the listed items may be used and only one of the items in the list may be needed. The item may be a particular object, thing, or category. In other words, “at least one of” means any combination of items or number of items may be used from the list, but not all of the items in the list may be required. For example, “at least one of item A, item B, and item C” may mean item A; item A and item B; item B; item A, item B, and item C; or item B and item C. In some cases, “at least one of item A, item B, and item C” may mean, for example, without limitation, two of item A, one of item B, and ten of item C; four of item B and seven of item C; or some other suitable combination.

As used herein, a system, apparatus, structure, article, element, component, or hardware “configured to” perform a specified function is indeed capable of performing the specified function without any alteration, rather than merely having potential to perform the specified function after further modification. In other words, the system, apparatus, structure, article, element, component, or hardware “configured to” perform a specified function is specifically selected, created, implemented, utilized, programmed, and/or designed for the purpose of performing the specified function. As used herein, “configured to” denotes existing characteristics of a system, apparatus, structure, article, element, component, or hardware which enable the system, apparatus, structure, article, element, component, or hardware to perform the specified function without further modification. For purposes of this disclosure, a system, apparatus, structure, article, element, component, or hardware described as being “configured to” perform a particular function may additionally or alternatively be described as being “adapted to” and/or as being “operative to” perform that function.

Although specific embodiments of the invention have been described and illustrated, the invention is not to be limited to the specific forms or arrangements of parts so described and illustrated. The scope of the invention is to be defined by the claims appended hereto and their equivalents.

What is claimed is:

1. A sleep aid system comprising:
a computing device;
a plurality of monitoring sensors coupled to the computing device and configured to monitor a user before and during sleep of the user;
one or more stimulation devices coupled to the computing device, wherein the one or more stimulation devices are configured to produce one or more stimuli; and
wherein the computing device processes inputs from the plurality of monitoring sensors and activates the one or more stimulation devices to produce the one or more stimuli based on the processed inputs.

2. The sleep aid system of claim 1, wherein the plurality of monitoring sensors comprises a wearable device on the user, wherein the wearable device is connected to the computing device.

3. The sleep aid system of claim 2, wherein the plurality of monitoring sensors comprises a temperature sensor, an audio reception device, and a camera.

4. The sleep aid system of claim 3, wherein the one or more stimulation devices includes a speaker configured to emit sounds.

5. The sleep aid system of claim 4, wherein the one or more stimulation devices includes a scent dispenser configured to emit scents.

6. The sleep aid system of claim 5, wherein the one or more stimulation devices includes one or more lights configured to produce different sequence or intensity of lights for the user.

7. The sleep aid system of claim 6, wherein the computing device is connected to a thermostat and the computing device is further configured to adjust the temperature based on the inputs from the plurality of monitoring sensors.

8. The sleep aid system of claim 7, wherein the computing device is further configured to adjust the lights based on the inputs from the plurality of monitoring sensors.

9. The sleep aid system of claim 8, wherein the computing device is further configured to adjust audio output from the speaker based on the inputs from the plurality of monitoring sensors.

10. The sleep aid system of claim 1, wherein the plurality of monitoring sensors are configured to record and monitor the sleep pattern of the user and match the sleep pattern to monitored conditions in a room where the user sleeps.

11. The sleep aid system of claim 10, wherein the monitored conditions include a temperature and an amount of light in the room.

12. The sleep aid system of claim 1, wherein the computing device is configured to run a sequence of stimuli matched to a sleep pattern of the user.

13. The sleep aid system of claim 1, wherein the computing device sends data collected from the user to a cloud connected computing device, wherein the cloud computing device includes an artificial intelligence (AI) algorithm that monitors the inputs and stimuli and determines a personalized stimuli sequence for the user.

14. The sleep aid system of claim 13, wherein the computing device is configured to run a sequence of stimuli to wake a user from sleep.

15. A sleep aid system, comprising:
a computing device;
a plurality of monitoring sensors coupled to the computing device and configured to monitor a user before and during sleep of the user, wherein the plurality of monitoring sensors includes wearable device on the user, a temperature sensor, an audio reception device, or a camera;
one or more stimulation devices coupled to the computing device, wherein the one or more stimulation devices are configured to produce one or more stimuli; and;
wherein the computing device processes inputs from the plurality of monitoring sensors and activates the one or more stimulation devices to produce the one or more stimuli based on the processed inputs.

16. The sleep aid system of claim 15, further comprising a cloud computing device connected to the computing device.

17. The sleep aid system of claim 16, wherein the computing device sends data collected from the user to the cloud connected computing device, wherein the cloud computing device includes an artificial intelligence (AI) algorithm that monitors the inputs and stimuli and determines a personalized stimuli sequence for the user.

18. The sleep aid system of claim 15, wherein the computing device is connected to a thermostat and the computing device is further configured to adjust the temperature based on the inputs from the plurality of monitoring sensors.

19. The sleep aid system of claim 15, wherein the one or more stimulation devices includes one or more lights configured to produce different sequence or intensity of lights for the user, and wherein the computing device is further configured to adjust the lights based on the inputs from the plurality of monitoring sensors.

20. The sleep aid system of claim 15, wherein the one or more stimulation devices includes a speaker configured to emit sounds, and wherein the computing device is further configured to adjust audio output from the speaker based on the inputs from the plurality of monitoring sensors.
